(19) United States
(12) Patent Application Publication
(10) Pub. No.: US 2004/0167398 A1
(43) Pub. Date: Aug. 26, 2004

(54) Combined emission tomography and computer tomography unit

(76) Inventors: Thomas Flohr, Uehlfeld (DE); Otto Sembritzki, Wachenroth (DE)

Correspondence Address: HARNESS, DICKEY & PIERCE, P.L.C., P.O.BOX 8910, RESTON, VA, 20195, US

(21) Appl. No.: 10/721,347

(22) Filed: Nov. 26, 2003

(30) Foreign Application Priority Data
Nov. 29, 2002 (DE) 10256075.7

(51) Int. Cl. A61B 5/05

(52) U.S. Cl. 600/427

(57) ABSTRACT

A combined emission tomography and computer tomography unit (ET/CT unit) is for imaging an object to be examined, and a method is for producing ET images of an object to be examined with the aid of a combined ET/CT unit. In the method, account is taken during production of the ET images of the spatial distribution, measured by the CT, of the attenuation coefficients in the measuring field of the CT in order to correct the measured ET image. Attenuation coefficients of the object that are disposed outside the measuring field are determined by extrapolation of detector channels and likewise used to correct the ET images.

The present application hereby claims priority under 35 U.S.C. §119 on German patent application number DE 102 56 075.7 filed Nov. 29, 2002, the entire contents of which are hereby incorporated herein by reference.

### FIELD OF THE INVENTION

An embodiment of the invention generally relates to a combined emission tomography and computer tomography unit (ET/CT unit) for imaging an object to be examined. Preferably, it relates to one having a scintillation detector and an evaluation unit for recording radiation that is emitted from the object to be examined, is generated by positron emitters and that is emitted from an object to be examined. The computer tomography unit (CT) preferably produces a distribution of the attenuation coefficients of the object to be examined in the examined region, and the measured data of the emission tomography unit (ET) is preferably directed by the distribution, recorded by the CT, of the attenuation coefficients in the object to be examined.

An embodiment of the invention also generally relates to a method for producing ET images of an object to be examined with the aid of a combined ET and CT unit, account being taken during production of the ET images of the spatial distribution, measured by the CT, of the attenuation coefficients in the measuring field of the CT in order to correct the measured ET image.

In the meaning of embodiments of the invention, the generic term emission tomography is to be understood as a method in which the radioactive substance is introduced into the object to be examined and the emission thereof is measured by corresponding detectors.

### BACKGROUND OF THE INVENTION

Specific methods include single proton emission computer tomography (SPECT) and proton emission tomography (PET). In contrast thereto, is the method of computer tomography (CT), in the case of which the object to be examined is transilluminated from an external radiation source, preferably an X-ray tube, in order to produce tomographic images that reproduce the distribution of the attenuation coefficients in the object to be examined. With regard to this method, reference may be made to the relevant chapter of “Bildgebende Systeme für die medizinische Diagnostik” [“Imaging Systems for Medical Diagnostics”], Editor Heinz Morneburg, 3rd edition, 1995.

In order to improve the quality of PET images, it is known from the publication by P. E. Kinahan, et al., “Attenuation Correction for a Combined 3D PET/CT Scanner”, Med. Phys. 25(10), 2046-2053 (October 1998) to make use of the information relating to the attenuation coefficients of an object to be examined that stems from CT images, and to carry out corrective calculations in the PET image.

When producing ET images that are corrected on the basis of CT measurements with reference to the distribution of attenuation coefficients not otherwise taken into account, the problem arises that in the case of known combined ET/CT units and methods, no account is taken in the corrective calculation of the PET image of attenuation coefficients of the object to be examined that are situated outside the measuring field of the CT.

### SUMMARY OF THE INVENTION

It is therefore an object of an embodiment of the invention to find a combined ET/CT unit and a method for producing PET images that also permit, by way of attenuation correction, improved ET images of objects that exceed the CT measuring field.

To date, the attempt has fundamentally been to design the CT measuring field of a combined ET/CT unit to be as large as possible such that even large objects to be examined are covered as fully as possible by this measuring field in order to supply optimum data for the corrective calculation of the ET image. However, geometric limits encountered here that can be overcome only by rendering the unit decidedly over dimension are thus extremely expensive.

The inventor has now realized that extending the CT reconstruction region beyond the CT measuring field, as is disclosed in patent application DE 100 26 566 A1, the entire contents of which are incorporated herein by reference, can also be used for a combined PET/CT unit. The known patent application from the applicant describes a CT unit that solves the so-called “large object” problem, that is to say the projection of the object to be examined beyond the measuring field. This is done by virtue of the fact that the reconstruction region is extended beyond the measuring field by extrapolating beyond the measuring field the data obtained in the measuring field such that even despite the incomplete measured data record outside the measuring field are good approximate values available for the attenuation coefficients of the object to be examined that are present there.

In this application, measuring field is understood as a circular measuring region inside a CT, the center of which lies at the fulcrum of a gantry, and whose circumference is touched tangentially by the outer fan rays of the fan-shaped radiation beam of the CT radiation source. This measuring field thus constitutes the set of points that are covered by the fan beam at all angles of rotation of the gantry.

Within the meaning of embodiments of the invention, the reconstruction region is to be understood as the region that is covered by the image reconstruction known per se, when producing CT images.

The content of DE 100 26 566 A1 referring to the extension of the reconstruction region beyond the measuring region, and the specific execution thereof by extrapolation methods, and the content of the publication by P. E. Kinahan, et al., “Attenuation Correction for a Combined 3D PET/CT Scanner”, Med. Phys. 25(10), 2046-2053 (October 1998), with reference to the correction method of PET images on the basis of known distributions of attenuation coefficients in the object to be examined, are hereby incorporated herein by reference in their entirety, into the disclosure of this document.

In accordance with the basic idea outlined above, the inventor proposes an improvement of the ET/CT unit known per se. The known combined ET/CT unit for imaging an object to be examined has:

an ET unit with a scintillation detector, and an evaluation unit for recording the radiation emanating from the object to be examined, and

a CT unit with at least one radiation source rotating about a system axis, from which there emanates a fan-shaped radiation beam that scans a measuring field and supplies together with a detector system output signals from which the distribution of the attenuation coefficients of the object to be examined can be reconstructed with reference to a reconstruction field,

means provided that correct the detected data of the ET unit by means of the distribution, examined by the CT, of the attenuation coefficients in the object (P) to be examined.

An improvement of an embodiment of the invention resides in that the CT unit uses a reconstruction field that is larger than the measuring field.

The result of this is that regions outside the CT measuring field are opened up without a change in the dimensions of the unit, and are used to calculate the correction of the ET images with reference to the attenuation coefficients.

The ET/CT unit in the CT part preferably includes a device(S) for extrapolating the measured data for the region of the reconstruction field that is situated outside the measuring field in order to extrapolate data of the reconstruction field that are situated outside the measuring field.

It can also be advantageous in this case when the CT includes device(s) that obtain the data for the region of the reconstruction field that is situated outside the measuring field by extrapolating cut projections.

If unnecessary computing time is to be suppressed, the CT can include devices that detect cut projections and extrapolate data, referring to the region of the reconstruction field situated outside the measuring field, for detected cut projections.

In order to avoid artifacts, it is further possible for the ET/CT unit to device(s) that that subject the extrapolated data to smoothing.

The measuring field and the reconstruction field can have a circular contour and be arranged concentrically with one another, the radiation beam of the CT emanating from a focus of the radiation source that moves on a circular path about the system axis.

In this case, the measuring field of the CT advantageously covers the circular region that is circumscribed by the outer rays of the radiation beam of the CT, and the reconstruction field together with the measuring field also comprises at least the region of the object to be examined that goes beyond the measuring field, preferably a circular region that covers the entire object.

The CT can have as radiation source an X-ray source emanating X radiation, preferably having a rotating anode.

In a simple design, a dedicated detector system can be used respectively for the ET and the CT part of the combination unit, the detector system of the CT part being designed here with the CT radiation source such that it can rotate about the system axis of the unit.

Furthermore, in a particularly compact design of the ET/CT unit the radiation detectors of CT and ET can be identical, it also being possible, if appropriate, for the evaluation unit of the ET, and the central processor of the CT to be combined in a single central processor.

It may be pointed out that all the above named functional device(s) preferably, but not exclusively, can include appropriate computer programs or program modules that execute the outlined functions when they are run.

In accordance with the basic idea of an embodiment of the invention outlined above, the inventors also propose an improved method that serves to produce ET images of an object to be examined with the aid of a combined ET and CT unit, account being taken during production of the ET images of the spatial distribution, measured by CT, of the attenuation coefficients in the measuring field of the CT in order to correct the measured ET image. The improvement resides in this case in that attenuation coefficients of the object that are disposed outside the measuring field are determined by extrapolation of detector channels and likewise used to correct the images, it being preferred to use extrapolation of cut projections or point reflection or linear extrapolation.

It is possible in a supplementary fashion to subject the extrapolated data to smoothing in order to achieve suppression of artifacts.

In the inventive ET/CT unit, the dedicated scintillation detector, preferably covering 360°, can be used for the CT part, and a dedicated detector rotating with a CT radiation source can be used for the CT part. If appropriate, a multi-row detector can also be used as CT detector.

In a further improvement and in order to scale down the system and reduce the redundant portions of a unit, it is also proposed to use a common detector to detect ET radiation and CT radiation. It is possible in this case to use a revolving 360° detector that is operated sequentially either as an ET detector with the CT radiation source switched off, or as CT detector with the CT radiation source switched off.

Since the attenuation coefficients determined differ in the CT method from the attenuation coefficients to be applied for the ET radiation, it is possible to convert from the measured CT attenuation coefficients to the attenuation coefficients to be expected with reference to the PET radiation at its level of 511keV. It is possible in this case to detect, for example, regions with tissue, bone, etc., and typical conversion factors can thereupon be applied in each case to the CT attenuation coefficients.

### BRIEF DESCRIPTION OF THE DRAWINGS

The present invention is described in more detail with reference to two exemplary PET/CT units with the aid of the figures and the present invention will become more fully understood from the detailed description of preferred embodiments given hereinbelow and the accompanying drawings, which are given by way of illustration only and thus are not limitative of the present invention, and wherein:

FIG. 1: shows a combined PET/CT unit with separate detector systems;

FIG. 2: shows a combined PET/CT unit with a common detector system for PET and CT.

### DETAILED DESCRIPTION OF THE PREFERRED EMBODIMENTS

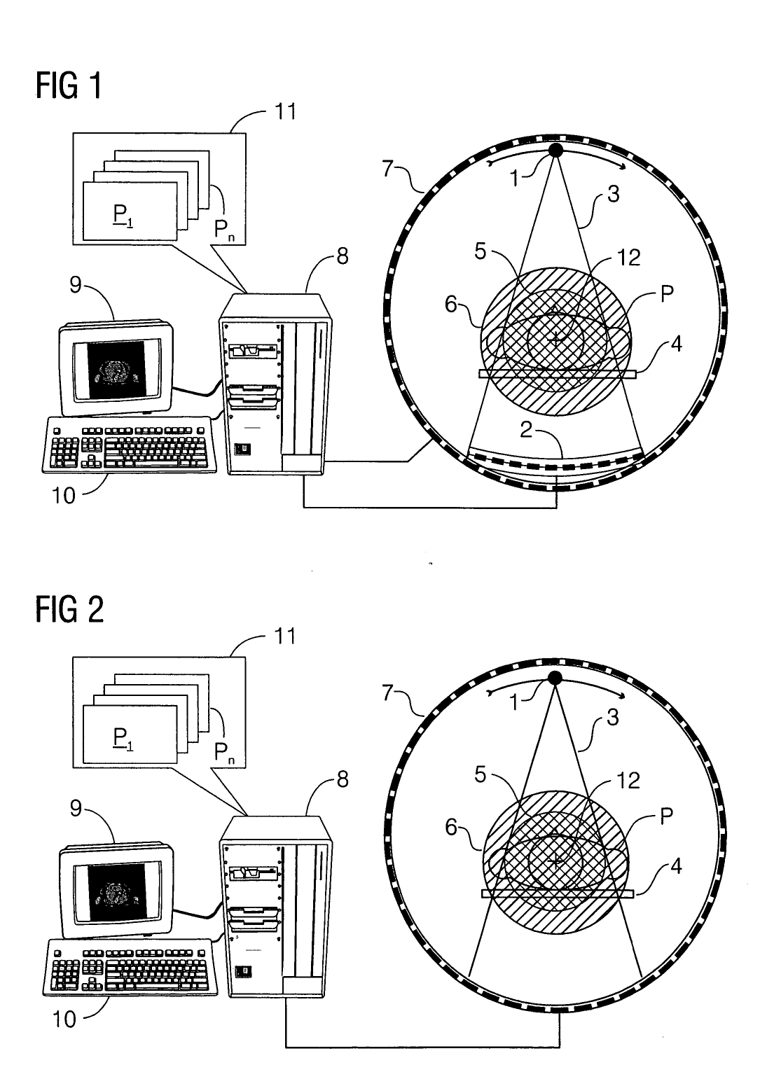

FIG. 1 shows a schematic illustration of a combined PET/CT unit with two separate detector systems 7 and 2, arranged offset in the direction of the system axis 12, for the PET and CT parts of the tomography.

The CT part of the unit has a gantry that can be rotated about the system axis 12 and to which there belongs a radiation source 1 and the detector system 2, including one or more rows. The radiation source 1 radiates a beam fan 3 with the aid of which a patient P or some other object to be examined and is lying on the examination bed 4 is scanned.

According to an embodiment of the invention, the distribution of the attenuation coefficients is reconstructed here—as described in detail in DE 100 26 566 A1—over a reconstruction field 6 that is larger than the direct measuring field 5. The direct measuring field 5 corresponds in this case to the circle that is formed about the axis of rotation of the gantry as center when the circumference of the circle is limited by the beam fan 3. The measuring field 5 is thus formed as a set of all points that are penetrated at each angle of rotation of the gantry by the beam fan, and thus having a completed 180° measured data record for calculating CT images.

The expansion of the reconstruction field beyond the measuring field is performed by extrapolating the measured data beyond this region, it being possible, for example, to apply a virtual expansion of the detector by a specific number of measurement channels, or an extrapolation of the measured data in this region. It is possible in this case to use, for example, a linear extrapolation over a specific number of outlying channels and measured values thereof, or a reflection at the last channel. In addition, in order to improve the method it is possible to undertake a smoothing of the data, in particular toward the edge region, in order to avoid a formation of artifacts.

After a reconstruction of the distribution of the attenuation coefficients has been determined over the reconstruction field 6 in the way according to an embodiment of the invention—with reference to the radiation of the CT radiation source 1, for example 120 keV X-radiation—these attenuation coefficients thus determined are used in a way known per se to infer the attenuation coefficients to be assumed for the 511 keV γ radiation occurring in the PET method. It is possible, for example, in this case for specific regions in the object to be examined to be assigned a different composition such as tissue, bone, etc., in order to find the best possible conversion factors from the CT attenuation coefficients to the PET attenuation coefficients.

If distribution of the attenuation coefficients of the PET radiation is known, this attenuation distribution can be taken into account appropriately on determining the PET image in order to suppress the absorption effects of the object to be examined that are occurring.

For this purpose, the area that is to be examined in the patient P is displaced in the direction of the system axis in order to use the scintillation detector 7 in a known way to scan with the PET method the region previously scanned with the CT method.

In the unit illustrated, all the computing methods are performed in a common evaluation unit and central processor 8 that is connected both to the CT detector 2 and to the PET detector 7 and whose various functions are taken over by the schematically illustrated programs P1-Pn. A keyboard 10 that can, of course, also be supplemented by further manual input units such as a mouse, joystick or the like is provided for inputting to and controlling the unit. The visual output is performed by the display screen 9, which is illustrated in representative fashion for other output media as well, such as a printer, video printer or the like.

It is now possible on the basis of the reconstruction region 6 enlarged by comparison with the previously customary measurement region still to obtain PET images with attenuation correction over the entire region of the object to be examined even in the case of objects to be examined that project beyond the measurement region of the CT, or to achieve an improved accuracy in this region respectively.

A more compact variant of the inventive combined PET/CT unit is illustrated in FIG. 2. By contrast with the unit of FIG. 1, this tomograph has a common detector 7 that is responsible both for the dosimetry of the CT examination and for the detection of the positron decay events in the PET examination.

It is advantageous in this case that because of its 360° design, the detector 7 is no longer absolutely constrained to rotate with the CT radiation source, it also being possible to carry out simultaneous measurements with the CT and PET methods, since this requires only the separation of the detected radiation events in terms of their energy content.

The illustrated designs of the detectors each exhibit single-row detectors, but multi-row detector systems or planar detectors are also possible with reference both to the CT system and to the PET system. In particular, it may be pointed out that an embodiment of the invention can be used with arbitrary combinations of ET and CT units without departing from the scope of the invention, but in particular with the two combinations of PET with X-ray CT and SPECT with X-ray CT.

It goes without saying that the abovementioned features of the invention can be used not only in the respectively specified combination, but also in other combinations or on their own without departing from the scope of the invention.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

what is claimed is:

1. A combined emission tomography and computer tomography unit (ET/CT unit) for imaging an object to be examined, comprising:
a scintillation detector and an evaluation unit for recording radiation emitted from the object to be examined;
a computer tomography unit (CT) including at least one radiation source rotating about a system axis, from which a fan-shaped radiation beam emanates that scans a measuring field and supplies, together with a detector system, output signals from which the distribution of the attenuation coefficients of the object to be examined are reconstructable with reference to a reconstruction field; and
means for correcting the detected data of the emission tomography unit (ET) using the distribution, examined by the CT, of the attenuation coefficients in the object to be examined, wherein the reconstruction field is larger than the measuring field.

2. The ET/CT unit as claimed in claim 1, wherein the CT includes means for extrapolating the measured data for the region of the reconstruction field that is situated outside the measuring field in order to extrapolate data of the reconstruction field that are situated outside the measuring field.

3. The ET/CT unit as claimed in claim 2, wherein the CT includes means for obtaining the data for the region of the reconstruction field that is situated outside the measuring field by extrapolating cut projections.

4. The ET/CT unit as claimed in claim 1, wherein the CT includes means for detecting cut projections and extrapolating data, referring to the region of the reconstruction field situated outside the measuring field for detected cut projections.

5. The ET/CT unit as claimed in claim 2, wherein the CT includes means for subjecting the extrapolated data to smoothing for the purpose of artifact reduction.

6. The ET/CT unit as claimed in claim 1, wherein the measuring field and the reconstruction field include a circular contour and are arranged concentrically with one another.

7. The ET/CT unit as claimed in claim 6, wherein the radiation beam of the CT emanates from a focus of the radiation source that is moveable on a circular path about the system axis.

8. The ET/CT unit as claimed in claim 1, wherein the CT includes, as a radiation source, an X-ray source emitting X radiation.

9. The ET/CT unit as claimed in claim 1, wherein the measuring field of the CT covers the circular region that is circumscribed by the outer rays of the radiation beam of the CT, and the reconstruction field together with the measuring field also includes at least the region of the object to be examined that goes beyond the measuring field.

10. The ET/CT unit as claimed in claim 1, wherein the CT part includes a dedicated radiation detector.

11. The ET/CT unit as claimed in claim 1, wherein the radiation detectors of the CT and ET are identical.

12. The ET/CT unit as claimed in claim 1, wherein the ET part of the unit is a PET, and further comprising means for determining coincident γ radiation that is generated by positron decay events.

13. The ET/CT unit as claimed in claim 1, wherein the ET part of the unit is a SPECT.

14. A method for producing ET images from an object to be examined with the aid of a combined ET and CT unit, comprising:
correcting a measured ET image using, during production of the ET images of the spatial distribution measured by the CT, attenuation coefficients in a measuring field of the CT, wherein the attenuation coefficients of the object to be examined, that are disposed outside the measuring field, are determined by extrapolation of detector channels.

15. The method as claimed in claim 14, wherein extrapolation of cut projections is used.

16. The method as claimed in claim 14, characterized wherein point reflection is used as an extrapolation method.

17. The method as claimed in claim 14, characterized wherein linear extrapolation is used as an extrapolation method.

18. The method as claimed in claim 14, wherein at least the extrapolated data are subjected to smoothing for the purpose of artifact reduction.

19. The method as claimed in claim 14, wherein different detectors are used to detect ET radiation and CT radiation.

20. The method as claimed in claim 14, wherein a common detector is used to detect ET radiation and CT radiation.

21. The method as claimed in claim 14, wherein the attenuation coefficients determined in the CT method are converted to the attenuation coefficients to be expected with reference to ET radiation.

22. The method as claimed in claim 14, wherein the PET method is used as ET method.

23. The method as claimed in claim 14, wherein the SPECT method is used as ET method.

24. The ET/CT unit as claimed in claim 2, wherein the CT includes means for detecting cut projections and extrapolating data, referring to the region of the reconstruction field situated outside the measuring field for detected cut projections.

25. The ET/CT unit as claimed in claim 1, wherein the measuring field of the CT covers the circular region that is circumscribed by the outer rays of the radiation beam of the CT, and the reconstruction field together with the measuring field also includes at least a circular region that covers the entire object.

26. The method as claimed in claim 15, wherein at least the extrapolated data are subjected to smoothing for the purpose of artifact reduction.

27. The method as claimed in claim 16, wherein at least the extrapolated data are subjected to smoothing for the purpose of artifact reduction.

28. The method as claimed in claim 17, wherein at least the extrapolated data are subjected to smoothing for the purpose of artifact reduction.

29. The method as claimed in claim 14, wherein a scintillation detector is used to detect ET radiation and CT radiation.
